(19) United States
(12) Patent Application Publication
(10) Pub. No.: US 2006/0234996 A1
(43) Pub. Date: Oct. 19, 2006

(54) Novel crystalline form of ezetimibe and processes for the preparation thereof

(76) Inventors: Itai Adin, Beer-Sheva (IL); Carmen Iustain, Beer-Sheva (IL)

Correspondence Address: Martin D. Moynihan;PRTSI, Inc., P.O. Box 16446, Arlington, VA, 22215, US

(21) Appl. No.: 11/402,962

(22) Filed: Apr. 13, 2006

Related U.S. Application Data
(60) Provisional application No. 60/671,094, filed on Apr. 14, 2005.

(51) Int. Cl. A61K 31/397; C07D 205/02

(52) U.S. Cl. 514/210.02; 540/200

(57) ABSTRACT

A novel crystalline form of Ezetimibe and processes for the preparation thereof are disclosed.

### RELATED APPLICATION

This application claims the benefit of U.S. Provisional Patent Application No. 60/671,094, filed on Apr. 14, 2005, the content of which is incorporated herein by reference.

### FIELD OF THE INVENTION

The present invention relates to solid state chemistry and more particularly to a novel crystalline form of Ezetimibe exhibiting improved properties and to processes for preparing it.

### BACKGROUND OF THE INVENTION

Ezetimibe (3R,4S)-1-(4-fluorophenyl)-3-[(3S)-3-(4-fluorophenyl)-3-hydroxypropyl]-4-(4-hydroxyphenyl)-2-azetidinone (I) is useful as hypocholesterolemic agent in the treatment and prevention of atherosclerosis.

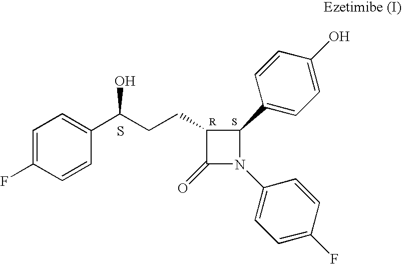

Ezetimibe belongs to a family of azetidinone compounds that are useful in lowering cholesterol levels and/or in inhibiting the formation of cholesterol-containing lesions in mammalian arterial walls. Examples for other compounds in this family are N-sulfonyl-2-azetidinone, which is disclosed in U.S. Pat. No. 4,983,597 and ethyl 4-(2-oxoazetidin-4-yl)phenoxy-alkanoates, which are disclosed by Ram et al. in Indian Journal of Chemistry, 29B:1134-1137, 1990.

Ezetimibe pharmaceutical use is disclosed in U.S. Pat. No. 5,767,115.

Its mechanism of action is different from other cholesterol-reducing drugs because it does not inhibit cholesterol synthesis in the liver or increase bile-acid excretion. Instead, Ezetimibe reduces blood cholesterol levels by inhibiting the absorption of cholesterol by the small intestine. This distinct mechanism is complementary to that of HMG-CoA reductase inhibitors, which inhibit cholesterol synthesis.

Ezetimibe preparation is described in Example 6 of U.S. Pat. No. 5,846,966 (reissued as RE37,721 and referred to herein as the '966 patent). In the final step of the synthesis, 1-(4-fluorophenyl)-3(R)-[3(S)-(4-fluorophenyl)-3-hydroxypropyl]-4(S)-[4-(phenylmethoxy)phenyl]-2-azetidinone is dissolved in ethanol and hydrogenated over Pd/C. The slurry is filtered off and the resulting solution is evaporated to obtain the final product, which has a melting point of 164-166° C.

The inventors of the present invention have repeated the procedure of obtaining Ezetimibe according to the teachings of the '966 patent, by evaporating the ethanol solution as described in example 1 of the present invention, and a crystalline material was produced having a powder X-ray diffraction as depicted in FIG. 1.

Additional crystalline modifications of Ezetimibe are disclosed in Patent Application WO 2005/009955 (hereinafter the '955 application). Two crystalline modifications, originally referred to as H1 and H2, as well as an amorphous form are disclosed in the '955 application. However, the X-ray powder diffraction pattern of Form H2 is similar to the X-ray powder diffraction pattern depicted in FIG. 1.

Additional crystalline modifications of Ezetimibe are disclosed in Patent Application WO 2005/062897 (hereinafter the '897 application). Two crystalline modifications, originally referred to as Form I and Form II, as well as an amorphous form are taught in the '897 application. Ezetimibe Form I is obtained by precipitating the crude product from acidic methanol and Ezetimibe Form II is obtained by pressurizing the crude Ezetimibe, which can cause the formation of a disordered material.

### SUMMARY OF THE INVENTION

The present invention provides a novel crystalline form of Ezetimibe, denoted as Form E1, which produces a unique X-ray powder diffraction as depicted in FIG. 2 and in Table 1

According to one embodiment of the present invention, the crystalline Ezetimibe Form E1 produces a powder X-ray diffraction pattern with diffraction peaks at 7.9, 9.7, 12.0, 13.9, 14.7, 15.2, 15.7, 17.2, 18.6, 19.3, 19.8, 20.9, 21.2, 21.7, 22.7, 22.8, 23.4, 24.5, 25.3, 26.3, 27.0, 27.4, 28.2, 28.8, 29.5 and 30.0±0.2 degrees 2θ.

According to another embodiment of the present invention, the crystalline Ezetimibe Form E1 is further characterized by an infra-red spectrum as depicted in FIG. 3, wherein the bands at about 3433, 3252, 1595, and 1213 cm⁻¹ are most characteristic of this form.

According to yet another embodiment of the present invention, the crystalline Ezetimibe Form E1 is further characterized by a DSC curve as depicted in FIG. 4 and by a melting point of 162-165° C.

According to a preferred embodiment of the present invention, Ezetimibe Form E1 is prepared by a process comprising:
a) dissolving Ezetimibe in an organic solvent;
b) heating the mixture to reflux;
c) cooling for sufficient time period to enable crystallization; and
d) collecting the crystals by filtration and drying, optionally at elevated temperature.

According to yet another embodiment of the present invention, the organic solvent is selected from the group consisting of ethanol, ethyl acetate, hexane, acetonitrile, chloroform, N-methylaniline and mixtures thereof.

### BRIEF DESCRIPTION OF THE DRAWINGS

FIG. 1—Powder X-ray diffraction pattern of crystalline Ezetimibe obtained according to example 6 of the '966 patent;

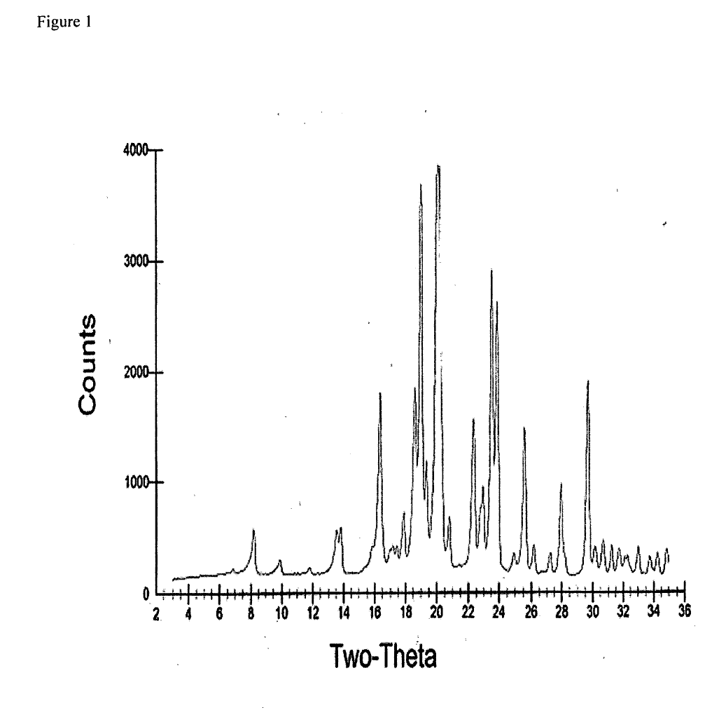

FIG. 2—Powder X-ray diffraction pattern of Ezetimibe Form E1;

FIG. 3—Infra-red spectrum of Ezetimibe Form E1;

FIG. 4—DSC curve of Ezetimibe Form E1.

### DETAILED DESCRIPTION OF THE PREFERRED EMBODIMENTS

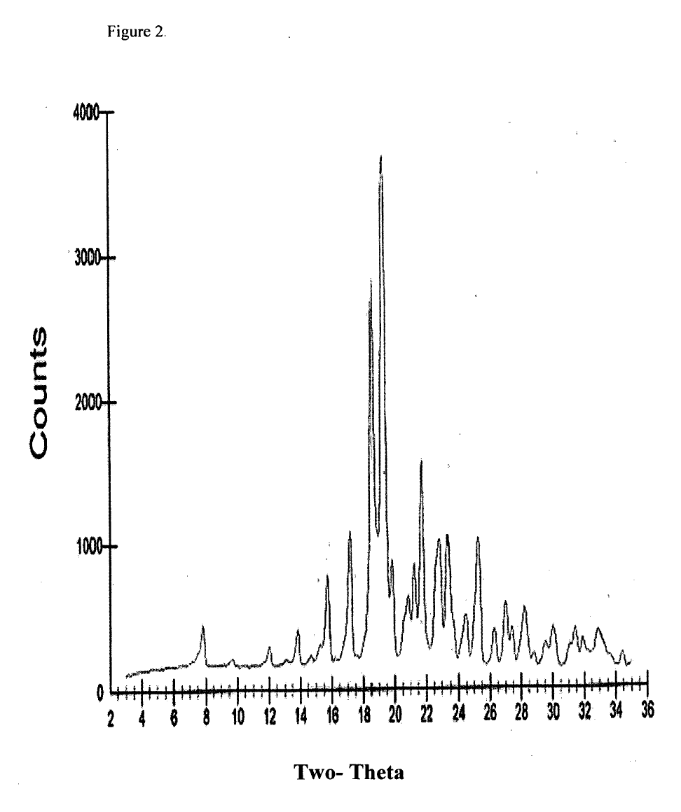

The present invention provides a novel crystalline form of Ezetimibe, denoted as Form E1, which produces a unique X-ray powder diffraction as depicted in FIG. 2 and in Table 1.

It is generally known that different crystalline forms possess different solid state properties, like hardness, bulk density, etc. The additional stable crystalline form provided therein, affords an important alternative option for the manufacture of Ezetimibe pharmaceutical dosage forms.

According to one embodiment of the present invention, the crystalline Ezetimibe Form E1 produces a powder X-ray diffraction pattern with diffraction peaks at 7.9, 9.7, 12.0, 13.9, 14.7, 15.2, 15.7, 17.2, 18.6, 19.3, 19.8, 20.9, 21.2, 21.7, 22.7, 22.8, 23.4, 24.5, 25.3, 26.3, 27.0, 27.4, 28.2, 28.8, 29.5 and 30.0±0.2 degrees 2θ.

Table 1 presents the powder X-ray diffraction peak positions and relative intensities of Ezetimibe Form E1.

TABLE 1

| Relative | | | |
|---|---|---|---|
| Intensity | Peak Position | Relative | Peak Position |
| (%) | (2θ deg) | Intensity (%) | (2θ deg) |
| 7 | 7.9 | 30 | 21.7 |
| 1 | 9.7 | 15 | 22.7 |
| 3 | 12.0 | 18 | 22.8 |
| 6 | 13.9 | 19 | 23.4 |
| 1 | 14.7 | 7 | 24.5 |
| 3 | 15.2 | 19 | 25.3 |
| 14 | 15.7 | 5 | 26.3 |
| 20 | 17.2 | 9 | 27.0 |
| 57 | 18.6 | 5 | 27.4 |
| 100 | 19.3 | 8 | 28.2 |
| 16 | 19.8 | 2 | 28.8 |
| 10 | 20.9 | 4 | 29.5 |
| 15 | 21.2 | 6 | 30.0 |

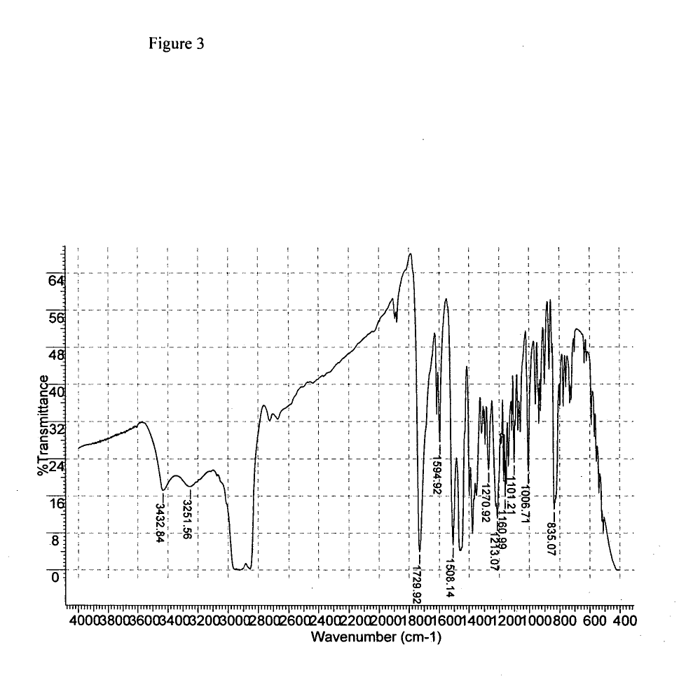

According to another embodiment of the present invention, the crystalline Ezetimibe Form E1 is further characterized by an infra-red spectrum as depicted in FIG. 3, wherein the bands at about 3433, 3252, 1595, and 1213 cm⁻¹ are most characteristic of this form.

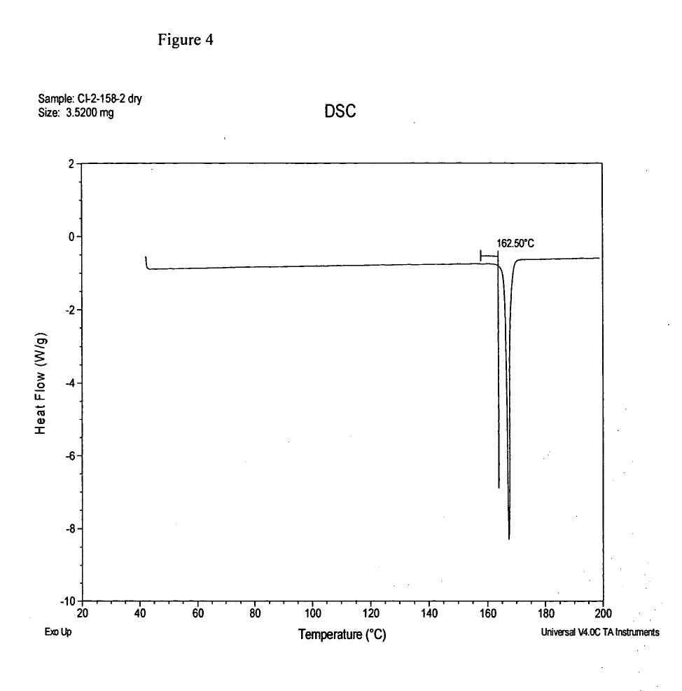

According to yet another embodiment of the present invention, the crystalline Ezetimibe Form E1 is further characterized by a DSC curve as depicted in FIG. 4 and by a melting point of 162-165° C.

According to a preferred embodiment of the present invention, Ezetimibe Form E1 is prepared by a process comprising:
a) dissolving Ezetimibe in an organic solvent;
b) heating the mixture to reflux;
c) cooling for sufficient time period to enable crystallization; and
d) collecting the crystals by filtration and drying, optionally at elevated temperature.

According to yet another embodiment of the present invention, the organic solvent is selected from the group consisting of ethanol, ethyl acetate, hexane, acetonitrile, chloroform, N-methylaniline and mixtures thereof.

In a preferred embodiment of the present invention, a mixture of ethanol/hexane is used as a solvent mixture to obtain Ezetimibe Form E1 according to the process described herein, wherein the preferred ratio between ethanol and hexane in the mixture is 1/3.3 v/v, and the weight/volume ratio between Ezetimibe and the ethanol/hexane 1/3.3 v/v mixture is at least 0.0038, preferably 0.046.

In another preferred embodiment of the present invention, a mixture of hexane/ethyl acetate is used as a solvent mixture to obtain Ezetimibe Form E1 according to the process described herein, wherein the preferred ratio between hexane and ethyl acetate in the mixture is 1/3 V/V, and the ratio between Ezetimibe and the hexane/ethyl acetate mixture is at least 0.0125, preferably 0.187.

In yet another preferred embodiment of the present invention, acetonitrile is used as a solvent to obtain Ezetimibe Form E1 according to the process described herein, wherein the weight/volume ratio between Ezetimibe and acetonitrile is at least 0.017 preferably 0.33.

In yet another preferred embodiment of the present invention, a mixture of hexane/chloroform is used as a solvent mixture to obtain Ezetimibe Form E1 according to the process described above, wherein the preferred ratio between hexane and chloroform in the mixture is 1/4.2 v/v, and the weight/volume ratio between Ezetimibe and the hexane/chloroform mixture is at least 0.0015, preferably 0.02.

In yet another preferred embodiment of the present invention, N-methylaniline is used as a solvent to obtain Ezetimibe Form E1 according to the process described herein, wherein the weight/volume ratio between Ezetimibe and N-methylamine is at least 0.017, preferably 0.233.

Table 2 summarizes the experimental methods for preparing Ezetimibe Form E1.

TABLE 2

| Example | | |
|---|---|---|
| Number | Solvent | Description of the experimental conditions |
| 2 | Ethanol/hexane | Ezetimibe (1.2 g) was dissolved in 26 ml of |
| | mixture | ethanol/hexane mixture. The solution was heated to |
| | | reflux and left to cool down to 25° C. The resulting |
| | | crystals (0.55 g) were filtered off and dried at |
| | | 50° C. under vacuum |
| 3 | Ethyl acetate/ | Ezetimibe (1.5 g) was dissolved in 8 ml of |
| | hexane mixture | hexane/ethyl acetate mixture. The solution was heated |
| | | to reflux, and left to cool down to 25° C. The resulting |
| | | crystals (1.08 g) were filtered off and dried at 50° C. |
| | | under vacuum |
| 4 | acetonitrile | Ezetimibe (2 g) was dissolved in 6 ml of acetonitrile. |
| | | The solution was heated to reflux, and left to cool |
| | | down to 25° C. The resulting crystals (0.98 g) were |
| | | filtered off and dried at 50° C. under vacuum |
| 5 | Chloroform/hexane | Ezetimibe (1 g) was dissolved in 42 ml of chloroform, |
| | mixture | and 10 ml hexane was added. |
| | | The solution was heated to reflux, and left to cool |
| | | down to 25° C. The resulting crystals (0.6 g) were |
| | | filtered off and dried at 50° C. under vacuum |
| 6 | N-methylaniline | Ezetimibe (1.4 g) was dissolved in 6 ml of N-methyl- |
| | | aniline. The solution was heated to reflux, and left to |
| | | cool down to 25° C. The resulting crystals (0.56 g) |
| | | were filtered off and dried at 50° C. under vacuum. |

### EXAMPLES

General Description of the Equipment:

X-ray diffraction data were acquired using a PHILIPS X-ray diffractometer model PW1050-70. System description: K₁=1.54178 Å, voltage 40 kV, current 28 mA, diversion slit=1°, receiving slit=0.2 mm, scattering slit=1° with a Graphite monochromator. Measurements of 2θ values typically are accurate to within ±0.2 degrees. Experiment parameters: pattern measured between 2θ=3° and 2θ=30° with 0.05° increments; count time was 0.5 second per increment.

Infra-red spectra were run on Nicolet Fourrier-transform infra-red spectrometer model Avatar 360, with Omnic software version 5.2. All samples were run as KBr disks. The current infra-red measurements are accurate to within 4 cm⁻¹.

Differential scanning calorimetry (DSC) measurements were run on TA instruments model Q1000, with Universal software version 3.88. Samples were analyzed inside crimped 40 μl Aluminum pans. Heating rate for all samples was 10° C./min.

### Comparative Example 1

Ezetimibe (2.0 g) was dissolved in 8 ml of ethanol. The solution was evaporated to dryness using a rotary evaporator at about 60° C.

### Example 2

In a 50 ml three necked round bottom flask equipped with a reflux condenser, a thermometer and a magnetic stirrer, Ezetimibe (1.2 g) was dissolved in 26 ml of 1/3.3 ethanol/hexane mixture. The solution was heated using an oil bath to reflux, and left to cool down to 25° C. The resulting crystals (0.55 g) were filtered off and dried at 50° C. under vacuum.

### Example 3

In a 50 ml three necked round bottom flask equipped with a reflux condenser, a thermometer and a magnetic stirrer, Ezetimibe (1.5 g) was dissolved in 8 ml of 3/1 ethyl acetate/hexane mixture. The solution was heated using an oil bath to reflux, and left to cool down to 25° C. The resulting crystals (1.08 g) were filtered off and dried at 50° C. under vacuum.

### Example 4

In a 50 ml three necked round bottom flask equipped with a reflux condenser, a thermometer and a magnetic stirrer, Ezetimibe (2 g) was dissolved in 6 ml of acetonitrile. The solution was heated using an oil bath to reflux, and left to cool down to 25° C. The resulting crystals (0.98 g) were filtered off and dried at 50° C. under vacuum.

### Example 5

In a 100 ml three necked round bottom flask equipped with a reflux condenser, a thermometer and a magnetic stirrer, Ezetimibe (1.0 g) was dissolved in 42 ml of chloroform. Hexane (10 ml) was added and the solution was heated using an oil bath to reflux, and left to cool down to 25° C. The resulting crystals (0.6 g) were filtered off and dried at 50° C. under vacuum.

### Example 6

In a 50 ml three necked round bottom flask equipped with a reflux condenser, a thermometer and a magnetic stirrer, Ezetimibe (1.4 g) was dissolved in 6 ml of N-methylaniline. The solution was heated using an oil bath to reflux, and left to cool down to 25° C. The resulting crystals (0.56 g) were filtered off and dried at 50° C. under vacuum.

What is claimed is:

1. A crystalline solid comprising Ezetimibe Form E1 characterized by powder X-ray diffraction substantially as depicted in FIG. 2, with diffraction peaks at 7.9, 9.7, 12.0, 13.9, 14.7, 15.2, 15.7, 17.2, 18.6, 19.3, 19.8, 20.9, 21.2, 21.7, 22.7, 22.8, 23.4, 24.5, 25.3, 26.3, 27.0, 27.4, 28.2, 28.8, 29.5 and 30.0±0.2 degrees 2θ.

2. The crystalline Ezetimibe Form E1 of claim 1, further characterized by infra-red spectrum substantially as depicted in FIG. 3, wherein the bands at about 3433, 3252, 1595, and 1213 cm⁻¹ are most characteristic of this form.

3. The crystalline Ezetimibe Form E1 of claim 1, further characterized by DSC curve substantially as depicted in FIG. 4 and by a melting point of 162-165° C.

4. A process for preparing the crystalline Ezetimibe Form E1, comprising:
a) dissolving Ezetimibe in an organic solvent;
b) heating the mixture to reflux;
c) cooling for sufficient time to enable crystallization; and
d) collecting the crystals by filtration and drying, optionally at elevated temperature.

5. The process of claim 4, wherein the solvent is selected from the group consisting of ethyl acetate, hexane, ethanol, acetonitrile, chloroform, N-methylaniline, and mixtures thereof.

6. The process of claim 5, wherein the solvent is a mixture of ethanol/hexane.

7. The process of claim 6, wherein the ratio between ethanol and hexane in the said mixture is 1/3.3 v/v.

8. The process of claim 7, wherein the weight/volume ratio between Ezetimibe and the ethanol/hexane 1/3.3 V/V mixture is about 0.046.

9. The process of claim 5, wherein the solvent is a mixture of hexane/ethyl acetate.

10. The process of claim 9, wherein the ratio between hexane and ethyl acetate in the mixture is 1/3 v/v.

11. The process according to claim 10, wherein the weight/volume ratio between Ezetimibe and the hexane/ethyl acetate mixture is about 0.19.

12. The process of claim 5, wherein the solvent is acetonitrile.

13. The process of claim 12, wherein the weight/volume ratio between Ezetimibe and acetonitrile is about 0.33.

14. The process of claim 5, wherein the solvent is a mixture of hexane/chloroform.

15. The process of claim 14, wherein the ratio between hexane and chloroform in the mixture is 1/4.2 v/v.

16. The process of claim 15, wherein the weight/volume ratio between Ezetimibe and the hexane/chloroform mixture is about 0.02.

17. The process of claim 5, wherein the solvent is N-methylamine.

18. The process of claim 17, wherein the weight/volume ratio between Ezetimibe and N-methylaniline is about 0.23.

19. A pharmaceutical composition comprising the crystalline solid comprising Ezetimibe Form E1 of claim 1 and pharmaceutically acceptable excipients and additives.
